(19) United States
(12) Patent Application Publication
(10) Pub. No.: US 2018/0325424 A1
(43) Pub. Date: Nov. 15, 2018

(54) Method for Estimating Thermal Ablation Volume and Geometry

(71) Applicant: Andrea Borsic, Torino (IT)

(72) Inventor: Andrea Borsic, Torino (IT)

(21) Appl. No.: 15/595,737

(22) Filed: May 15, 2017

(51) Int. Cl. A61B 5/107; A61B 34/10

(52) U.S. Cl. CPC A61B 5/1076; A61B 34/10; A61B 2018/00577; A61B 2034/105; A61B 2034/107

(57) ABSTRACT

This invention pertains a system and methods for ablation treatment of tissues. The invention aims to improve current models that allow predicting the volume and geometry of thermal ablations. Particularly the invention consists in a method that allows accounting for effects that occur when vapor that forms at the ablation site is able to seep in cavities that might encroach the ablation site and to deliver heat to the tissues of those cavities, creating an ablation geometry that is not described by current ablation models.

### STATEMENT REGARDING FEDERALLY SPONSORED RESEARCH OR DEVELOPMENT

The research activity leading to this patent application had been partially supported by the SBIR Phase I grant 1R43CA189515-01 awarded from the U.S. National Cancer Institute.

### FIELD OF INVENTION

The present disclosure pertains generally to systems and methods for interventional guidance of tissue ablation procedures.

### BACKGROUND OF THE INVENTION

The goal of the invention is to improve accuracy in the prediction of the volume and geometry of thermal ablations.

Thermal ablation technologies are used to treat tissues for therapeutic purposes. An example of application is treatment of cancer, where thermal ablation is used to necrotize malignant tissues in order to cure or manage the disease.

Two primary thermal ablation technologies exist: Radio Frequency Ablation (RFA) and Microwave Ablation (MWA). RFA is based on the application of Radio Frequency (RF) energy to the tissues by means of one or multiple contacting electrodes. MWA is based on the application of Micro Wave (MW) energy to the tissues by means of a contacting antenna. Both technologies cause a local increase in the temperature of tissues which ultimately causes the necrosis of a certain volume of tissues (treatment of tissues). If the volume of treated tissues encompasses all the tissues which are target of the procedure, the treatment is adequate. A single procedure might require multiple overlapping ablations to treat the whole volume of target tissues.

RFA and MWA can be applied in a minimally invasive fashion. Both RFA and MWA are, for example, used in percutaneous treatment of liver cancer, where a needle-shaped RFA electrode, or MWA antenna, are inserted, through the skin, into the volume of the tumor and used to treat the target tissues.

FIG. 1 shows, for example, and RFA electrode, where a needle-shaped cannula (101) is able to release in the tissues a number of metallic filaments which deploy in an umbrella-like fashion (102). These filaments are named “tines”, they are in electrical contact with tissues and allow encompassing and treating a larger volume of tissues.

Modeling of the physics taking place in RFA or MWA allows developing computer models that compute the temperatures in the tissues and the volume/geometry of tissues necrotized. Prediction of the volume/geometry of treated tissues is useful for interventional planning and for intraoperative guidance purposes, as discussed in the following.

In the context of interventional planning it is possible to use computer-generated ablation volume/geometry information to develop systems that superimpose to images of the patient, like Computed Tomography (CT), Magnetic Resonance Imaging (MRI), Ultrasound (US), or Positron Emission Tomography (PET), a computer generated representation of the estimated ablation volume/geometry for a particular ablation device and energy level. FIG. 2 shows for example a contrast-CT image revealing a tumor (201), and FIG. 3 shows the same contrast-CT image to which has been superimposed a computer generated model of an RFA electrode (301) and an indication of the volume which would be necrotized, represented as a semi-transparent surface (302).

Visualizations of this kind allow a physician to study which tissues would be treated by a particular ablation device, energy setting, and position/orientation of the device inside the body. The physician would be able to use such visualizations to plan the intervention, for example, by determining the single or multiple optimal positions/orientations of the ablation device inside the body, and levels of applied energy, which would result in the complete treatment of the target tissues.

In the context of intraoperative guidance visualizations similar to FIG. 3 would support physicians in evaluating of which tissues are treated and which not by a particular ablation directly in the operating room, offering a “see-and-treat” functionality.

As computer-generated ablation volumes/geometries would be used to inform physicians about which tissues would be necrotized during a procedure, accuracy of the models used to compute such volumes/geometries is of particular importance.

### CURRENT STATE OF THE ART

Modeling of thermal ablations is commonly based on the Bioheat equation (see Detailed Description of the Invention) which, given the distribution of RF or MW power applied to the tissues, determines the resulting temperatures in the tissues.

As during ablation tissue temperatures often exceed the evaporation temperature of water, the Bioheat equation is generally expanded to account for the effect of evaporation of water in the tissues. A model which represents the state-of-the-art in modeling evaporation has been proposed in [1].

As during a percutaneous ablation the vapor that forms is not able to leave the body (a needle-shaped ablation device is inserted in the tissues through the skin, and there is no path for the vapor to exit the body), evaporation models assume that the vapor which forms at the hottest points of the ablation site will diffuse in the porous matrix of tissues and condense at locations where the temperature of tissues is inferior to the evaporation temperature of water. In these models no vapor is therefore assumed to leave the body.

Any evaporation/condensation model therefore assumes that evaporation occurs in a certain region of tissues (hotter) and that condensation occurs at other regions (colder). Part of the model deals with determining which are the regions where evaporation occurs and which are the regions where condensation occurs.

In regions where evaporation occurs, heat is absorbed from tissues by the evaporating water, in regions when condensation occurs heat is delivered to tissues by the condensing vapor.

The model proposed in [1], for example, makes these assumptions:
1. The quantity of heat which is absorbed from tissues by the evaporation of water is released back to tissues in those regions where condensation occurs.
2. Evaporation occurs at any point is tissue where the temperature is greater than the evaporation temperature of water.
3. Condensation occurs in tissues which have a temperature between 80° C. and 60° C.

The first assumption of the model accounts for the fact that no vapor escapes from the body, and so the heat absorbed from evaporation is fully released back to the tissues by condensation. The second assumption simply states that evaporation occurs where the temperature is sufficiently high.

The third assumption is empirical and models that fact that vapor will diffuse from the hottest tissues where evaporation occurs to nearby tissues, at lower temperatures, where it will condense.

FIG. 4 illustrates regions where evaporation and condensation occurs, according to [1], for an RFA electrode. In this figure a model of an RFA electrode is formed by a shaft (401) and several tines (402). Three temperature 2D iso-lines, which are cross sections in the plane of the figure of corresponding 3D iso-surfaces, are shown. The isoline (403) indicates the temperature of 100° C. (temperature of evaporation of water in standard pressure conditions). The area enclosed by this isoline represents therefore the region of tissues where evaporation occurs. The isoline (404) is relative to the temperature of 80° C. and the isoline (405) is relative to the temperature of 60° C. The area enclosed between these two isolines (404 and 405) therefore, according to [1], is the region where condensation occurs.

### SUMMARY OF THE INVENTION

Through animal studies, in the context of liver ablation, we have discovered that the assumptions of evaporation models that are the current state-of-the-art, including [1], are not valid in circumstances where vapor generated by the ablation has access to physical paths in the tissues (e.g. ducts, tracks, fissures, holes) through which it can escape the ablation site. In these cases a certain amount of vapor will travel in these physical paths available to it, carrying and delivering heat to tissues in ways that depend on the physical geometry of these paths. The resulting heat distributions are not predicted by current evaporation/condensation models, which assume that vapor will only diffuse and condense in homogeneous tissues surrounding the ablation site.

We have observed the above phenomenon in at least two circumstances.

In a first circumstance, vapor forming at the ablation site travels in the track created in the tissues by the insertion of the ablation needle. Vapor propagates in the interstitial space between the needle and the tissues for a certain length along the needle (as described in the Detailed Description of the Invention). This phenomenon results in the heating of a cylindrical region around the ablation needle, something not accounted for by current evaporation models.

In a second circumstance, vapor forming at the ablation site, under the pressure created by the evaporation itself, will travel in liver fissures and transport heat along this interstitial space. The liver is composed of multiple lobes which are separate, but face each other. The interstitial space between two lobes is called a fissure. If the ablation site encroaches a fissure, the fissure provides a path for vapor to escape the local high-pressure; vapor will therefore travel in this space, delivering heat to the tissues that are facing the fissure. This results in an altered ablation volume/geometry which is not predicted by current computer models.

The invention consists in a method for improving current evaporation/condensation models to account for this phenomena. This improvement is to be made by redistributing, in the model, a portion of the overall heat released by vapor to those volumes or surfaces of ducts, tracks, fissures, holes in which vapor is able to travel. Current models assume only a diffusion of vapor in uniform tissues, without considering the presence of ducts, tracks, fissures, holes and the effects that derive from them when vapor travels in them.

Modeling the physical paths in tissues (ducts, tracks, fissures, holes) through which vapor can travel requires knowledge of their geometry, which is patient and procedure specific.

In the specific case of the tracks generated in tissues by the insertion of needle-shaped ablation devices, the length, position, and orientation of the track can be known from images of the patient capturing the device as deployed in the tissues, or by using surgical instrument tracking technologies (e.g. electromagnetic, such the NDI Aurora system, or optic, such as the Medtronic StealthStation), which would indicate the position/orientation of the needle-shaped device in the tissues.

In the specific case of liver fissures, as fissures are thin interstitial spaces and they offer low contrast to imaging. The spatial resolution of the imaging modalities available is not sufficient to capture them, and so their geometry cannot be known by imaging means. To model the geometry of liver fissures we propose to use deformable liver models, which include information about the location of the fissures in a standard anatomy, and which are adapted to the liver of the patient—as captured from images. The fissures' geometry in the deformed model is assumed to be indicative of the true position of the fissures in the patient, and this information would be used in the evaporation/condensation model.

While a certain degree of uncertainty on the true location of the fissures in the patient might remain when using a deformable liver model to estimate this information, the proposed approach would allow to account for the effect of fissures, an aspect not accounted for in any way in the current clinical practice, which results in the necrotization of tissues beyond the indications available to physicians, as discussed in the Detailed Description of the Invention.

Alternatively, in order to make imaging of liver fissures possible, we propose to modify the ablation needle in such a way that it is able to inject high contrast liquids, gasses, or powders in the tissues, where these liquids, gasses, or powders would penetrate fissures that encroach the ablation site, and render them visible in the images—thanks to the high contrast properties of the injected liquids, gases, powders. This would allow to capture the geometry of the fissures by imaging, and to use this information for modeling the effects of vapor traveling in those fissures.

### BRIEF DESCRIPTION OF THE DRAWINGS

FIG. 1 depicts an example of a common commercial RFA electrode design which deploys tines.

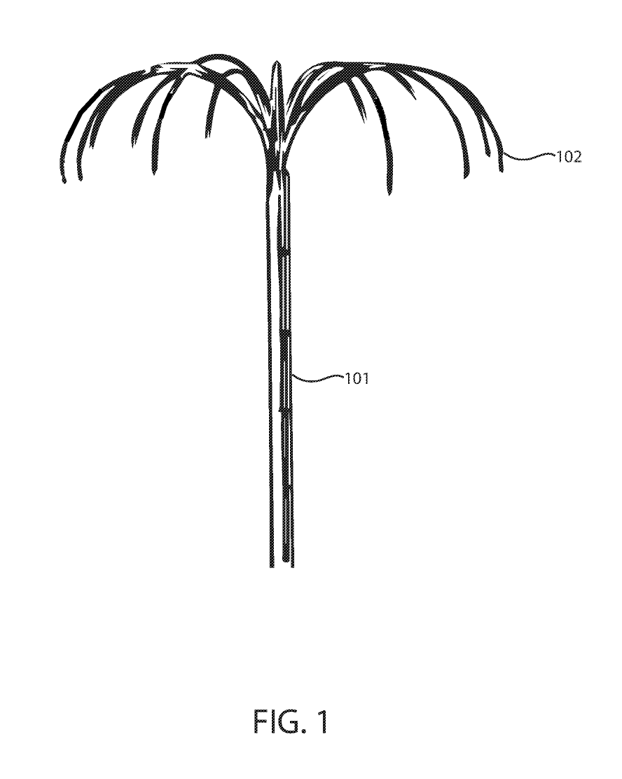

FIG. 2 depicts a CT image where a tumor is visible as an enhancing area of lighter color.

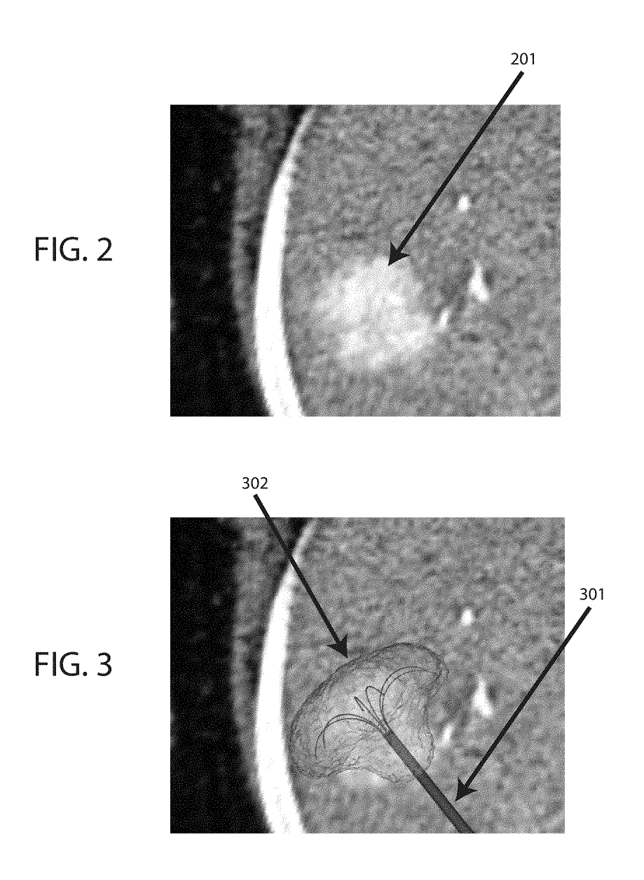

FIG. 3 depicts the CT image of FIG. 2 with superimposed the volume/geometry of an ablation as computed with a computer model simulating RFA physics.

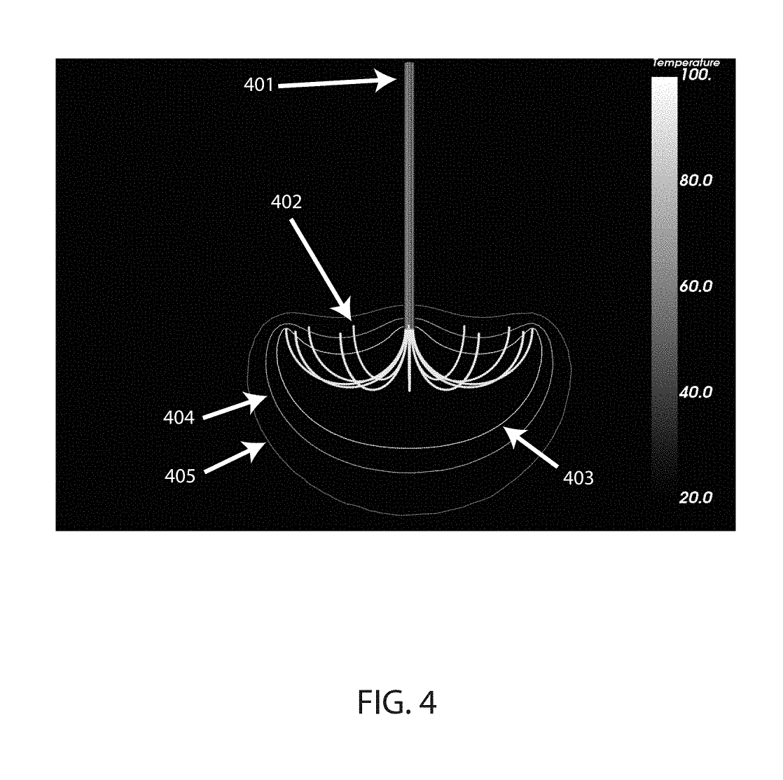

FIG. 4 depicts an RFA electrode and the 100° C., 80° C., and 60° C. iso-temperature lines for the temperature field established in the tissues.

FIG. 5 depicts a CT image of the liver of a pig immediately after RFA. In the image is visible the RFA electrode inserted in the tissues and it is possible to appreciate the fact that vapor has formed in a region in the proximity of the needle of the electrode.

FIG. 6 depicts a computer model of an RFA electrode and a cylindrical volume around the needle of the electrode where a quantity of thermal power is delivered to account for the thermal power which is transported in this region of tissues by vapor traveling along the needle interstitial space.

FIGS. 7A, 7B, 7C depicts the geometry of necrotized tissues as captured in a liver RFA pig study (FIG. 7A), the equivalent volume generated by the current state-of-the-art evaporation model (FIG. 7B), and a model which is a particular embodiment of the present invention (FIG. 7C). This last model produces a predicted ablation volume which is dose to the true ablation volume captured by in-vivo imaging.

FIG. 8 depicts an ablation site where the presence of a liver fissure allows vapor to use this physical path to escape the ablation site and to necrotize tissues that are not expected to be involved in the ablation. This results in an ablation volume which departs from the information provided by the RE ablation needle manufacturer and from the ablation geometry produced by current models.

FIG. 9 depicts harvested pig liver tissues after RFA, showing that vapor has traveled in a fissure and has necrotized tissues on the two sides of the fissure.

FIG. 10 depicts two lobes of the liver separated by a fissure which is encroached by an RFA electrode. In the figure are indicated facing surfaces of the fissure where heat from condensing vapor is redistributed to account for the effects of vapor traveling in the fissure and heating tissues.

FIGS. 11A, 11B, 11C depict respectively: a deformable liver model (FIG. 11A); a CT image of a liver (FIG. 11B); the deformable liver model fitted to the liver in the CT image (FIG. 11C).

TABLE 1 reports data showing the model used to compute the ablation geometry, which is a particular embodiment of this invention, is able to improve the accuracy of the predicted volume/geometry, reducing the maximum error from 9.4 mm to 5.2 mm, on average across six analyzed ablations.

### DETAILED DESCRIPTION OF THE INVENTION

### Detailed Background Description

Modeling in thermal ablation is commonly based on the Bioheat equation [1]. As during ablation tissue temperatures in RFA and MWA often exceed 100° C., the Bioheat equation is generally expanded to account for the effect of evaporation of water in the tissues, as for example as in [1], leading to (1)

$$ρ c \frac{\partial T}{\partial t} = ∇ \cdot k ∇ T + {Q}_{PWR} + {Q}_{PERF} + {Q}_{E} \qquad ( 1 )$$

where ρ is the tissue density, c is the tissue thermal capacitance, T is the temperature in the tissues, t is time, k is the tissue thermal conductivity, Q_{PWR} is the dissipated power density resulting by the applied RF or MW power, Q_{PERF} is the heat density lost to perfusion, Q_{E} is the heat density lost to evaporation, or gained from condensation of vapor in the tissues. The quantities ρ, k, c, T, Q_{PWR}, Q_{PERF}, Q_{E} are functions of space (scalar fields), while, of course, t, the variable indicating time, is a scalar quantity. The quantities ρ, k, c are properties of tissues, which can be considered as given fixed values, or as functions of temperature themselves. The term Q_{PWR} models the RF or MW power density applied by the RF/MW needle and dissipated in the tissues. This is a distributed heat source. The perfusion term Q_{PERF} is a power density that models the fact that a quantity of heat is lost to the capillary bed in the region around the ablation site. This loss occurs as the temperature of blood (37° C.) is lower than the temperature reached by tissues during ablation, therefore an amount of heat will flow from the heated tissues to the capillaries, and later this heat will be taken away by the blood flow (perfusion). This term is therefore a distributed heat sink term.

The Q_{E} term is a power density that describes instead the effects of evaporation/condensation of water in the tissues. During an ablation, temperatures in excess of 100° C. are reached in the tissues at locations in the proximity of the needle. At these locations a certain fraction of the water present in the tissues evaporates. During the state change from liquid to gas the water absorbs a quantity of heat called latent heat. Vapor will diffuse in the tissues, under the pressure it generates, and as it meets tissues at a lower temperatures it will condense, and release in those lower temperature tissues the latent heat. The term Q_{E} is therefore negative (heat sink) at locations where evaporation occurs (heat is absorbed from tissues) and positive (heat source) at locations where the vapor condenses (heat is released to tissues). The magnitude of the term Q_{E} will depend, at any point in the tissues, by the rate of at which water is evaporating or condensing at that location.

Overall, this mechanism is therefore a heat transport mechanism mediated by vapor, where heat is subtracted from tissues where evaporation occurs and re-delivered to tissues where vapor condenses.

Modeling this heat transport mechanism requires therefore determining in which regions of the simulated domain evaporation occurs and in which regions condensation occurs.

Regions in which evaporation occurs are those where the local tissues temperature reaches the evaporation temperature of water (100° C., or a similar temperature which might be determined by the local pressure).

In these regions the term Q_{E} can be expressed as:

$${Q}_{E} = - α \frac{dW}{dt} \qquad ( 2 )$$

where α is the latent heat constant for water and W is the tissue water density, and t is time. Equation (2) applies to any point in the tissues where evaporation occurs, and Q_{E} is the thermal power density absorbed from tissues. The total thermal power absorbed from tissues from evaporation at any instant in time, which we label Q_{E}_{_}_{TOT}, is found by integrating (2) over a volume that encapsulates all the tissues where evaporation occurs, and can be expressed as:

$${Q}_{{E}_{—} TOT} = - \int α \frac{dW}{dt} d Ω \qquad ( 3 )$$

where Ω represents the region of tissues over which the integration is carried out.

To summarize: 1) evaporation occurs at points where the tissue temperature is greater than the evaporation temperature of water; 2) the thermal power density absorbed at any point in tissues from evaporation is (2); 3) the total thermal power absorbed from evaporation from all the tissues at any point in time is (3).

The evaporation/condensation models which are object of this invention are pertinent to percutaneous ablation, where vapor forming from evaporation of tissue water has no path to escape the body of the patient. It is assumed therefore that all the vapor that has formed will condense in tissues.

Determining the regions where vapor condenses requires determining how vapor diffuses in the tissues.

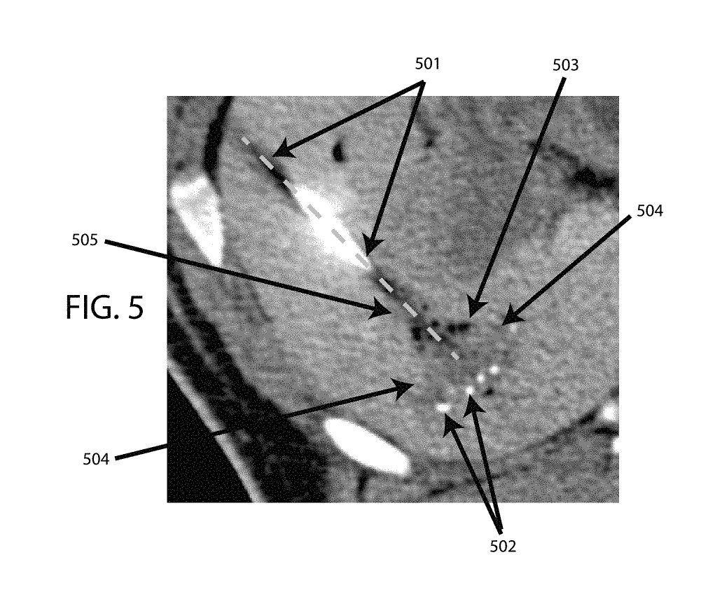

In the evaporation/condensation model [1], which constitutes the state-of-the-art, it is empirically assumed that at any time all the thermal power absorbed by evaporation (Q_{E}_{_}_{TOT}) will be re-distributed uniformly to tissues where the temperature is comprised between 60° C. and 80° C. These tissues are typically a region of tissues in the proximity of the ablation site, where condensation is likely to occur. FIG. 4 shows as an example an RFA electrode formed by a needle shaped cannula (401), and by a number of tines (402). The isolines for temperatures of 100° C., 80° C., and 60° C. are respectively (403), (404), and (405). Under model assumptions in [1] evaporation would occur in the volume enclosed by the 100° C. isoline, and condensation would occur in the volume enclosed between the 80° C. and 60° C. isolines.

The state-of-the-art evaporation model [1] can be summarized therefore as follows:
Evaporation region defined by T>100° C.;

$${Q}_{E} = - α \frac{dW}{dt}$$

Condensation region defined by 60° C.<T<80° C.; Q_{E}_{_}_{TOT}/Vol_{—60—80}
In any other region Q_{E}=0
   where Vol_{—60—80} is the volume of tissues with 60° C.<T<80° C., where condensation occurs.

The model proposed in [1], representing the state-of-the-art, allows therefore to define regions in tissues where evaporation or condensation occurs and to determine the value of Q_{E} in these regions, allowing to use this value of Q_{E} in (1) and to compute the temperatures in tissues subject to the effects of evaporation/condensation.

This evaporation/condensation model is empirical and works well where tissues are uniform.

### Invention Description

Vapor diffusion in tissues during thermal ablations is driven by the pressure that forms at the ablation site caused by the evaporation itself. In certain circumstances preferential paths (preferential to diffusion in tissues) might be available to vapor for traveling from points at a higher pressure to points at a lower pressure. These paths consist in interstitial spaces in the organs, in ducts, in tracks present in the tissues which encroach the ablation site, and which represent a possible escape path for the vapor. As vapor travels through these paths it will encounter tissues at temperatures inferior to the evaporation temperature and it will release heat to these tissues and condense. This results in a distribution of heat which is determined, in part, by the geometry of these paths where vapor travels. As a consequence the overall heat distribution around the ablation site can be quite different from the case where vapor simply diffuses in uniform tissues—as assumed by current evaporation/condensation models.

In general, when these paths are present, a certain proportion of all the generated vapor will travel in them and a certain proportion will continue to diffuse in the tissue because of their porosity. To model this, we split the term Q_{E}_{_}_{TOT} in two quantities. A quantity (1-b) Q_{E}_{_}_{TOT} of thermal power is redistributed to the tissues uniformly (the scalar b is in the range 0 to 1 and sets the amount of vapor that is redistributed uniformly) to those tissues having a temperature comprised between a lower and a higher specified threshold (e.g. 60° C. and 80° C.), modeling diffusion and condensation in tissues similarly to what proposed in [1]. A quantity b Q_{E}_{_}_{TOT} of power is instead redistributed along those surfaces, or in those volumes, representing the preferential paths where vapor travels and condenses (tracks, ducts, interstitial spaces, holes); this models the effects of heat which is transported and released by condensing vapor to those locations.

### Embodiment Example, Modeling Vapor Diffusion and Condensation Along the Needle Shaft

In the specific case of RFA and MWA, where percutaneous ablation devices are shaped like needles, we have observed in animal experiments that vapor seeps in the track created in the tissues by the insertion of the needle-shaped ablation device. Specifically vapor generated at the distal end of the device, where the ablation occurs, travels for a certain length along the interstitial space between the needle and tissues, following the needle track towards the proximal-end.

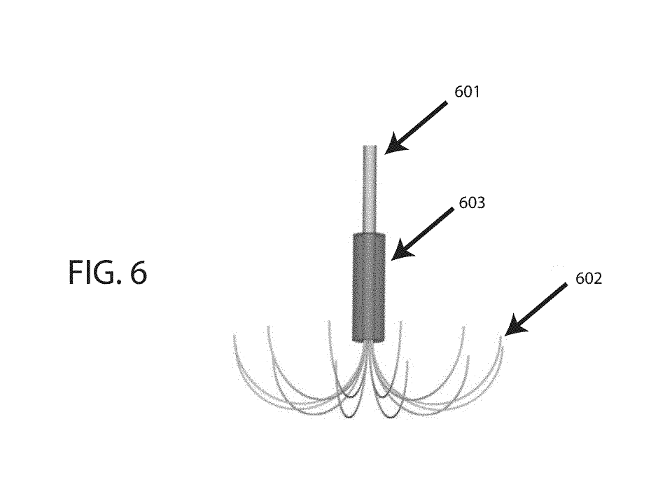

FIG. 5 shows, as an example, a CT image of percutaneous liver RFA in a pig, conducted under institutional IACUC approval. The image was collected immediately after RFA was performed. The dotted line (501) indicates the direction and position of the shaft of the needle electrode. The white dots (502) indicate the tines, which are intersecting the imaging plane, and appear as bright dots as they are metallic objects. The dark dots (503) indicate the presence of vapor in the proximity of the location where the shaft of the electrode meets the tines, which happens to be the hottest point in the ablation volume. The tissues necrotized be the ablation are visible in a slightly darker shade of gray compared to normal tissues. The bulk of the volume of necrotized tissues (504) is encompassed by the tines and it extends towards the shaft of the electrode, becoming narrower, and forming overall a triangular shape.

Necrotized tissues extend for some length along the shaft of the electrode (505). The presence of necrotized tissues extending along the shaft of the electrode is not expected, as the shaft is electrically insulated and thus does not actively heat the tissues. The presence of necrotized tissues is instead explained by the fact that vapor, under pressure, is able to penetrate the interstitial space between the shaft of the electrode and the tissues, and to follow this track for some length along the shaft of the electrode. The vapor that infiltrates this space delivers a certain amount of heat to tissues in this region and necrotizes them.

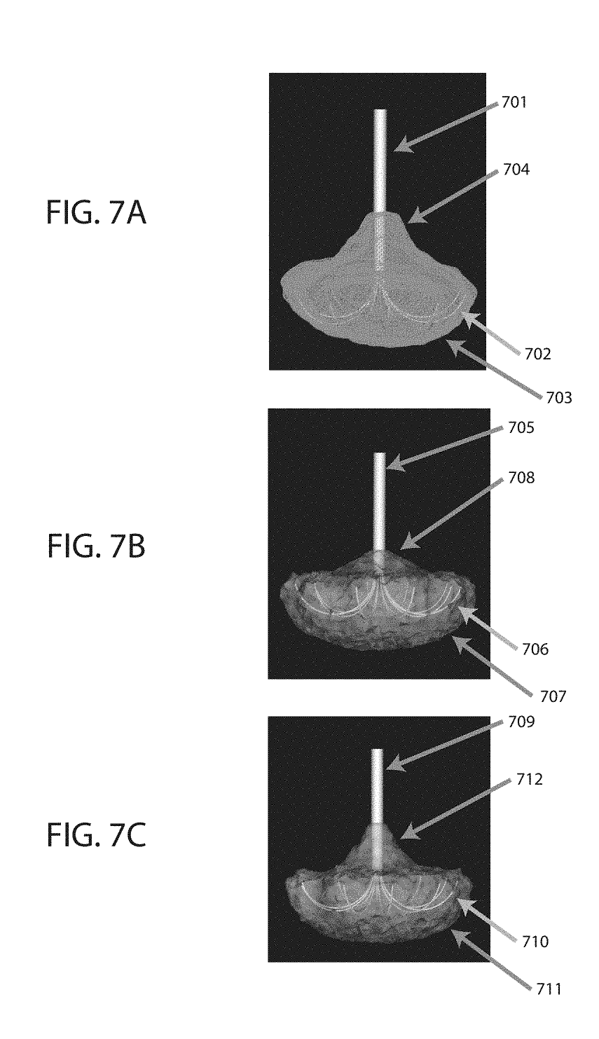

In order to account for this phenomenon, in a particular embodiment of the method proposed, where this specific case being discussed is modeled, we redistribute the power b Q_{E}^{Tot} uniformaly in a cylindrical region around the shaft of the electrode, accounting in this way for the heating that occurs in such region due to vapor traveling in the interstitial space between the electrode shaft and tissues. FIG. 6 shows a model of an RFA electrode formed by a shaft (601) and a number of tines (602). The darker cyclindrical volume (603) indicates the region where the power b Q_{E}^{Tot} is redistributed.

Use of this model improves the accuracy with which the abaltion volume can be predicted.

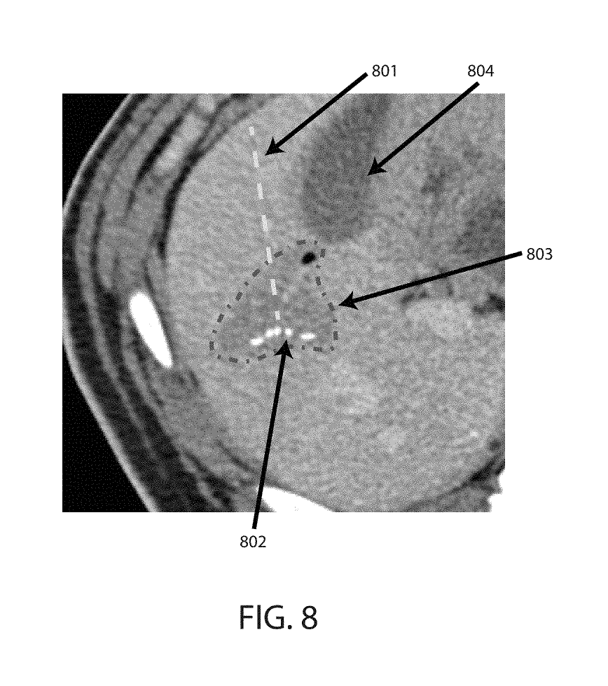

FIG. 7A shows the geometry of an abaltion performed in a pig liver. In the figure is visible a computer model of the RFA electrode, compraised of a shaft (701) and tines (702). The gray surface (703) represents the volume/geoemtry of the abaltion, which was captured by imaging with contrast-CT the liver immediately after the abaltion was performed, and by segmenting the tissues that appear to be necrotized. The volume of the abaltion extens for a certain length along the electrode shafy (704). For comparision purposes, FIG. 7B shows the abaltion volume estimated using the state-of-the-art model [1]. The predicted ablation volume/geoemtry is flatter at the top, and the volume of necrtized tissues does almost not extend upwords along the shaft of the electrode, point (708), the top of the abaltion volume, is lower than point (704).

FIG. 7C shows the same abaltion volume estimated with a model which is a particular embodiment of this invention. In FIG. 7C is visible a computer model of the RFA electrode compraised of a shaft (709) and tines (710). In this particular embodiment of the invention, the thermal power b Q_{E}^{Tot}, with b=0.2, was redistributed to a cylinder aligned to the electrode shaft, as in FIG. 6, with a diameter of 5 mm. The cylinder starts at the point where the shaft connects to the tines, and has a height of 2 cm. The remaining power (1-b) Q_{E}^{Tot} was distributed, as in [1], to tissues with temperatures between 60° C. and 80° C. Use of this model resulted in a more accurate prediction of the true abaltion volume and geometry compared to the state of the art. The top of the ablation volume, point (712), is now more elevated compared to the predictions from state-of-the-art models (point (708)), mimichking better the true abaltion geoemtry as captured by imaging in-vivo experiments, where the abaltion volume raises along the electrode shaft (704).

The improvements brought this invention, in the particular embodiment described above, were evaluated on six percutaneous liver ablations in pigs. Table 1 reports quantitative results. The first six rows of the table report results for each ablation site, and the last row of the table reports results averaged over the six ablation sites. The first column of the table, titled “Error Uniform Vap. Redist.”, reports the model error defined as the maximum distance between the surface of the ablation captured in-vivo by CT imaging and the surface of the ablation as computed with the model proposed in [1]. The column titled “Error Vap. Redist. Along Shaft” reports the error defined as the maximum distance between the surface of the ablation captured in-vivo by CT imaging, and the surface of the ablation as computed with the particular embodiment of this invention described above, where 20% (b=0.2) of thermal power from condensing vapor is distributed in a cylindrical region around the shaft, and all the remaining thermal power (80%) is distributed is the regions of tissues issues with a temperature between 60° C. and 80° C. The last column of the table reports the error reduction obtained by using the model which is a particular embodiment of this invention, compared to the model proposed in [1]. On average, across the 6 analyzed abaltion sites, the maximum error was reduced from 9.42 mm to 5.18 mm, a reduction of 44% using the particular embodiment of this invention.

### Embodiment Example, Modeling the Effect of Liver Fissures

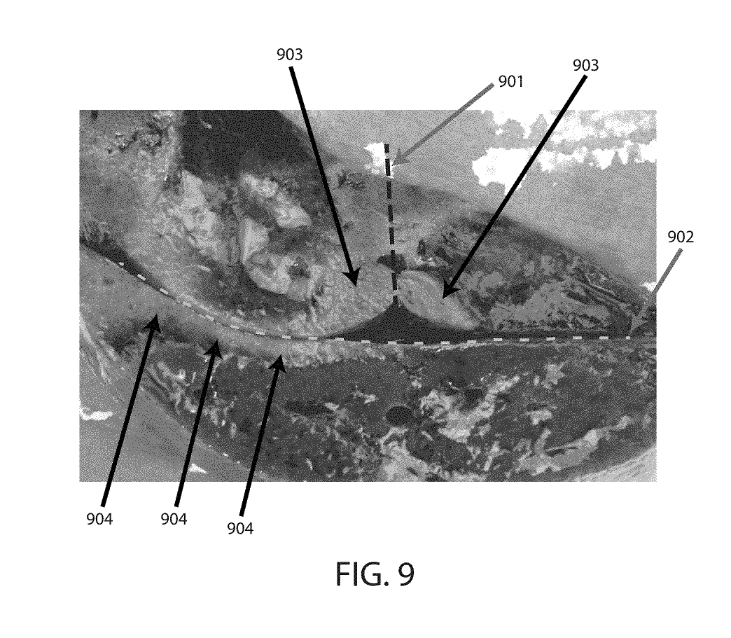

Fissures are present in the liver and they can offer a path to vapor generated during thermal ablations to escape the ablation site. As vapor travels in the fissure it delivers heat to the tissues facing the fissure and this results in a different ablation pattern than normally expected. FIG. 8 shows, as an example, a CT image of percutaneous liver RFA in a pig, conducted under institutional IACUC approval. The dashed line (801) indicates the position and orientation of the RFA electrode shaft (not visible as the imaging plane does not intersect it). The bright dots (802) indicate the position of the tines of the RFA electrode, as they intersect the imaging plane. The dash-dotted line (803) highlights the contour of the ablation, where necrotized tissues are visible in a darker shade of gray. The dark feature (804) indicates the gallbladder. In this specific ablation, a fissure that connects the ablation site to the gallbladder allowed vapor to escape the ablation site by traveling in the fissure towards the gallbladder. As a result the ablation is asymmetrical with respect to the electrode shaft, and the ablation has a “plume” shape that bends towards the gallbladder. The geometry of this ablation is not accounted for by models in the literature representing the state-of-the-art.

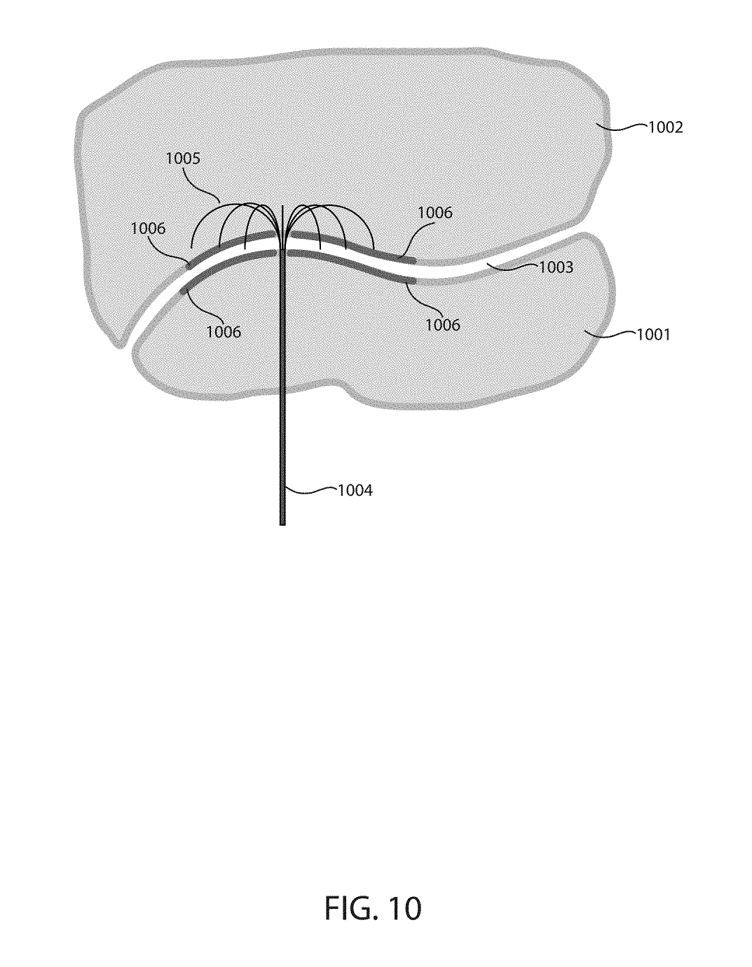

Post-mortem harvesting of the liver confirmed that vapor was able to necrotize tissues on the facing sides of the fissure. FIG. 9 shows the harvested liver which has been resected in a plane intersecting the ablation site. The dotted line (901) indicated the position and orientation of the RFA electrode shaft. The dotted line (902) highlights the fissure separating two lobes of the liver. Tissues that appear in lighter color in the proximity of the electrode shaft (903) are necrotized tissues. These tissues are normally expected to be necrotized by the ablation. Other necrotized tissues (904) are visible along the fissure and at a distance from the ablation site; these tissues have been necrotized by vapor that has formed in the fissure and that has delivered heat to the tissues facing the fissure, and normally not expected to be necrotized.

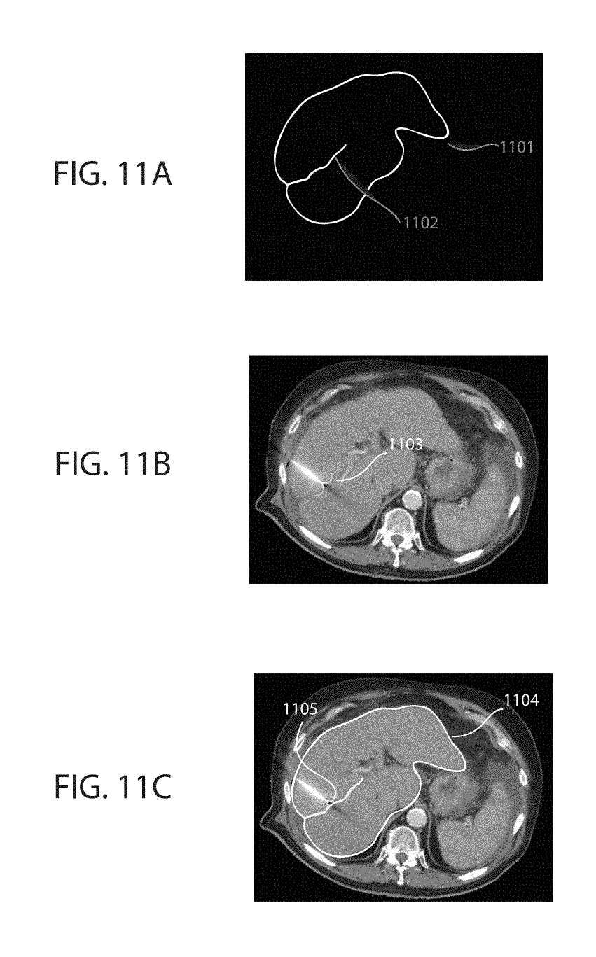

This particular situation can be modelled by redistributing a portion of the thermal power that vapor releases to tissues b Q_{E}^{Tot} to surface of the fissure. FIG. 10 shows an illustration where two lobes of a liver (1001) and (1002) are separated by a fissure (1003). An RFA electrode is used formed by a shaft (1004) and by a number of tines (1005). In this illustration the RFA electrode, and therefore the abaltion site, encroaches the fissure, and vapor forming at the abaltion site will seep in the fissure and heat the tissues facing the fissure. In order to model this phenomenon, a portion of the total thermal power absorbed by vapor b Q_{E}^{Tot} should be delivered to the surfaces of the fissure of the abaltion site (1006). This is similar to the approach discussed in the previous embodiment, where part of the heat of the condensing vapor was distributed in a cylindrical region of tissues (FIG. 6) to account for the effects of vapor.

In order to model the effects of vapor traveling in fissure according to the proposed embodiment, it is necessary to know the geoemtry of the fissure. Fissures are extremely thin interstitial spaces and they do not offer particular contrast, therefore they are not visible in medical images.

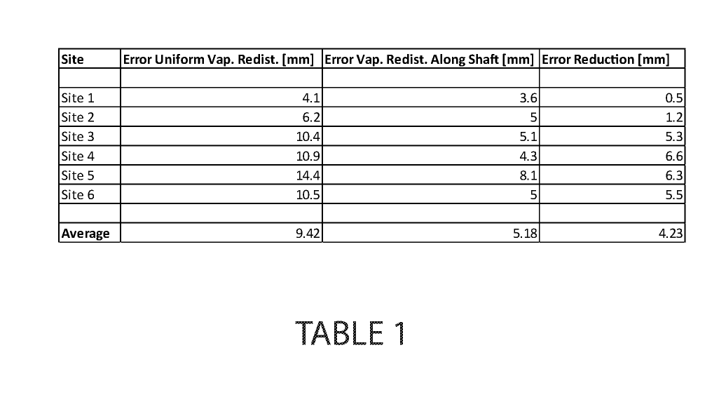

In a particular embodiment of this invention we propose to utilize a deformable liver model, as illustrated in FIGS. 11A, 11B, and 11C, to estimate the geoemtry of the fissures of the patient. In FIG. 11A is shown the contour of a deformable liver model (1101). The model includes a representation of the geoemtry of the fissure (1102). In FIG. 11B is shown a CT image of a human liver, where an RFA electrode has been deployed (1103). Fitting of the boundary (1101) of the deformable liver model to the true liver boundary (1104) available from CT images allows to update the shape of the liver model in such a way that the deformed model is a good representation of the patient's liver. This in turn allows to estimate whether the fissures of the model (1102) would encroach the abaltion site (1105). At this point at least two options are available. In the first option, the geometry of the fissures in the model is assumed to be a good representation of the true geoemtry of the fissures of the patient, and the volume/geoemtry of the abaltion is computed distributing an amount of thermal power b Q_{E}^{Tot} to these surfaces, while an amount of thermal power (1-b) Q_{E}^{Tot} is distributed to tissues simulating a diffusion of vapor in tissues. In a second option, the physician is simply warned that the current ablation site encroaces a fissure and that the abaltion geoemtry might be altered by the presence of the fissure.

Additionally to the approach of using a deformable model to estimate the geometry of the fissures, we propose to render them visible in images by injecting in them high-contrast media (e.g. high-contrast liquids, gases, powders) which could be delivered by the ablation device itself, and make the fissures visible, as this media penetrates the fissures and enhances their contrast.

### REFERENCES

[1] Yang, Deshan, Mark C. Converse, David M. Mahvi, and John G. Webster. “Expanding the bioheat equation to include tissue internal water evaporation during heating.” IEEE Transactions on Biomedical Engineering 54, no. 8 (2007): 1382-1388.

What is claimed is:

1. A method for estimating the volume and geometry of tissues necrotized by thermal ablations making use of models which account for the thermal effects of vapor traveling in ducts, interstitial spaces, holes, cavities present in the tissues and encroaching the ablations site, comprising the steps of:
computing the temperature in the tissues under the effect of the ablation power applied to the tissues, where this power might be, for example, of electrical or electromagnetic nature;
accounting for the thermal power absorbed from the tissues by the evaporation of water present in the tissues;
redistributing part of the thermal power absorbed from evaporation to the volumes and/or surfaces of said ducts, interstitial spaces, holes, cavities encroaching the ablations site, thus modeling the heating that vapor causes by traveling through those structures and/or by condensing in those structures;
updating the computed temperature based on the redistribution of heat to the volumes and/or surfaces of said ducts, interstitial spaces, holes, cavities encroaching the ablations site;
computing which tissues are necrotized by the ablation using a relationship that links, at least, but not limited to, the temperature in the tissues to the damage of tissues.

2. The method of claim 1 where part of the thermal power absorbed from evaporation is redistributed in a region along the shaft of a needle-shaped ablation device to model the heating effect of vapor that infiltrates the track in the tissues created by the insertion of the needle-shaped device, similarly to the particular embodiment shown in FIG. 6 where this region is cylindrical (603).

3. The method of claim 1 where part of the power absorbed from evaporation is redistributed to surfaces of liver fissures, to model the heating effect of vapor that infiltrates such fissures, similarly to the particular embodiment shown in FIG. 10 where these surfaces are indicated as (1006).

4. The method of claim 1 applied to the study or design of ablation devices.

5. The method of claim 1 applied in systems for treatment planning.

6. The method of claim 1 applied in systems for intraoperative guidance.

7. The method of claim 2 applied to the study or design of ablation devices.

8. The method of claim 2 applied in systems for treatment planning.

9. The method of claim 2 applied in systems for intraoperative guidance.

10. The method of claim 3 applied to the study or design of ablation devices.

11. The method of claim 3 applied in systems for treatment planning.

12. The method of claim 3 applied in systems for intraoperative guidance.

13. The method of using a deformable liver model carrying information about the anatomy of the liver fissures, and adapted to medical images of the patient, in order to estimate the true intracorporal position of the fissures in the patient from the adapted deformable liver model.

14. The method of injecting high-contrast media such as gases, powders, or liquids at the ablation site, where these gases, powders, or liquids might travel to ducts, interstitial spaces, holes, cavities present in the tissues and render these structures visible in medical images by virtue of enhancing contrast, and possibly by virtue of enlarging these gaps.
